(19) United States
(12) Patent Application Publication
(10) Pub. No.: US 2006/0246013 A1
(43) Pub. Date: Nov. 2, 2006

(54) Pump system for dispensing foaming eyelid cleanser

(75) Inventor: Nat Adkins JR., Richmond, TX (US)

Correspondence Address: D'AMBROSIO & ASSOCIATES, P.L.L.C., 10260 WESTHEIMER, SUITE 465, HOUSTON, TX, 77042, US

(73) Assignee: OCUSOFT, Inc.

(21) Appl. No.: 11/358,766

(22) Filed: Feb. 21, 2006

Related U.S. Application Data
(60) Provisional application No. 60/676,913, filed on May 2, 2005.

(51) Int. Cl. A61K 9/00; A61M 11/00

(52) U.S. Cl. 424/47; 128/200.23

(57) ABSTRACT

A non-propellant pump system for dispensing foaming eyelid cleanser comprising an eyelid cleanser, a container for the eyelid cleanser and a dispensing assembly for delivering the eyelid cleanser as a foam. The eyelid cleanser is a foamable aqueous solution comprising anionic surfactants, non-ionic thickener and emollients, amphoteric surfactants and at least one microbiological preservative. The dispensing assembly comprises a non-propellant pump for producing foam. The pump has a mixing chamber to combine the liquid cleanser with air, a foam producing element and an outlet passage for dispensing the foam.

This application claims the benefit of U.S. Provisional Application No. 60/676,913, filed May 2, 2005.

### FIELD OF THE INVENTION

The present invention relates to a pump system for dispensing foaming eyelid cleanser. More specifically, it relates to a non-propellant pump system for dispensing a foaming eyelid cleanser.

### BACKGROUND OF THE INVENTION

Ocular health refers to eyes as well as structures associated with the eyes, eyelids for example. The eyelids are important in over-all ocular health because they protect the eyes from dangers such as approaching objects or from airborne contaminants, such as pollen, dust particles or other foreign bodies. The eyelids contain essential glands; the lacrimal glands and meibomian glands that produce layers of tear film that are critical for healthy eyes. When an individual blinks, a new tear film is created and tears are distributed across the cornea to lubricate the surface of the eye. This blinking action also “flushes” foreign materials from the eye.

The eyelids, however, are subject to certain problems, which while very common, are none-the-less bothersome, especially for contact lens wearers, and may lead to other more serious complications. One complication is blepharitis. Blepharitis is a common chronic inflammation of the eyelids characterized by a scaly crust on the lid margins. The condition may be caused by a bacterial infection, or it may be allergic in origin or associated with seborrhea of the face and scalp. Treatment usually involves cleansing the eyelids on a regular basis to remove excess oil, debris, and desquamated skin that may be problematic.

Often associated with or secondary to blepharitis is a bacterial infection of the surface of the skin at the edge of the lid know as an internal hordeolum. Other such infections include external hordeolum, commonly referred to styes, which are infections of the tiny oil secreting meibomian glands along the edge of the eyelid, surrounding the eyelashes. A stye begins as a red, tender bump and usually fully develops within three days. Such conditions are accompanied by pain, redness and tenderness of the lid margins. Although styes are often recurring, regular cleansing of the eyelid margins can minimize such conditions. A second problem is a chalazion, which is an inflammation of the meibomian glands inside the eyelid. Chalazia typically grow slowly over 2-3 weeks and although they do not typically cause pain, they often require surgical intervention if left untreated.

With any of the above-described problems, as well as other medical complications, such as rosacea and seborrhea, proper eyelid hygiene with the use of an eyelid cleanser may minimize the severity of the outbreak, or prevent the problem altogether if caught early. Eyelid cleansers are also used for cleaning eyelashes, eyelids or the periocular area and may be used as a pre-operative scrub to help reduce the presence of harmful bacteria which may cause infection, inflammation, or even endophthalmitis in patients.

Eyelid cleansers are known in the art. An eyelid cleansing composition is taught by U.S. Pat. No. 4,904,698 to Adkins, Jr. et al, incorporated herein by reference in its entirely. Also, Adkins, Jr., U.S. Pat. No. 6,112,900, discloses a care kit for contact lens wearers that include products for routine contact lens care and eyelid cleansing. The eyelid cleanser is a non-irritating, antimicrobial solution that is soaked into a disposable pad.

Foam dispensing devices are known in the art. For example, Van der Heijden, U.S. Pat. No. 6,536,629, describes an aerosol for dispensing a liquid in the form of a foam. The device comprises a liquid pump with a pump chamber and a pump piston and an air pump with a pump chamber and a pump piston. A flexible sealing component, comprising two annular, resilient sealing lips are used to close off and open the inlet and the outlet of the air pump. The actuating head is pressed, so that the pistons of the air pump and the liquid pump are moved downwards, with the result that the volumes of the corresponding piston chambers are reduced and air and liquid are dispensed to a so-called mixing chamber, mixed, and the mixture passed into the outlet passage and leaves the dispenser in the form of a foam.

A drawback of the listed device and other types of applicators of an eyelid scrub are that the solution can often drip into the eyes or elsewhere. None of the above described devices, taken either singlely or in combination, is seen to describe the instant invention as claimed. What is needed is an external ocular cleansing product that can be dispensed in a foam delivery system so as to be pre-lathered, not requiring any outside physical or mechanical action to help prevent loss of cleanser. Further, the foam cleansing product may be used with reusable applicators, such as a washcloth as opposed to one-use disposable pads.

### DEFINITIONS

Surfactants are compounds which reduce surface tension when dissolved in water or water solutes; alternatively, surfactants are compounds that reduce interfacial tension between two liquids, or between a liquid and a solid.

A preservative is an agent that prolongs the useful life of a material.

A biocide is a substance that kills or inhibits the growth of microorganisms such as bacteria, molds, slimes, fungi, etc.

A chelating agent is a coordination compound in which a central metal ion is attached by coordinate links to two or more non-metal atoms in the same molecule.

All definitions are from Hawley's Condensed Chemical Dictionary, 13th Edition, John Wiley & Sons, Inc., 1997 or the American Heritage® Dictionary of the English Language, Fourth Edition, on line at bartleby.com.

### SUMMARY OF THE INVENTION

The present invention provides a pump system for dispensing foaming eyelid cleanser. This invention addresses the need for a system to dispense an ocular product that avoids dripping of the product into the eye itself. The foam cleaner with dispenser allows for proper care and cleaning of eyelids and the ocular area without irritation to the eyes caused by more flowable liquids that can accidentally enter the eye. Use of the pump system for dispensing foaming eyelid cleanser can reduce irritation and diseases of the ocular area. Another advantageous feature of the invention is that the pump system dispenses the eyelid cleaner as a foam, which cleans immediately, as opposed to a liquid surfactant, such as soap, which require mechanical lathering before being effective to clean. The preferred pump system is a non-aerosol system, thereby further reducing irritation to the ocular area.

Preferably, the eyelid cleanser dispensing system comprises an eyelid cleanser, a container for the eyelid cleanser and a dispensing assembly for delivering the eyelid cleanser as a foam to the user. The eyelid cleanser is a foamable liquid in an aqueous solution comprising an anionic surfactant, a non-ionic thickener and emollient, an amphoteric surfactant and at least one microbiological preservative. These include a polyoxyethylenesorbitan fatty acid ester, polyoxyethylene fatty acid ester, lauroamphocarboxy glycinate, sodium laureth-13 carboxylate, and PEG-15 tallow polyamine.

Further, the foamable eyelid cleanser comprises a surfactant mixture comprising PEG-80 sorbitan laurate, sodium trideceth sulphate, PEG-150 distearate, cocamidopropylhydroxy sultaine, lauroamphocarboxy glycinate, and sodium laureth-13 carboxylate.

The eyelid cleanser comprises the following compounds as a percentage of the total weight of the composition: the surfactant mixture present in a concentration of 7-10%, PEG-15 tallow polyamine present in a concentration of 0.1-0.5%, at least one preservative selected from the group consisting of Quaternium-15 and benzyl alcohol, present in a concentration of 0.1-0.5% and a chelating agent present in a concentration of 0-0.1%. The PEG-15 tallow polyamine is a tertiary surfactant and emollient. The remainder of the volume of the eyelid cleanser comprises water.

Advantageously, the eyelid cleanser dispensing assembly comprises a non-propellant pump for producing a foam. Chemical propellants frequently used aerosol dispersers can be irritating to the eye. The non-propellant pump used with the applicant's invention uses air to produce a foam. The non-propellant pump system is a dispensing system comprising a container for the liquid eyelid cleanser in combination with a non-propellant pump foam dispensing assembly. The pump has a mixing chamber to combine with air, a foam producing element and an outlet passage.

The foam producing element comprises one or more meshes for producing the foam, which are contained in the outlet passage. The mixing chamber comprises an actuating head with the outlet passage containing the meshes and a circumferential protective cap.

Preferably, a method for manufacturing a dispenser for a foaming eyelid cleanser comprises preparing an eyelid cleanser, placing the eyelid cleanser in a container; and attaching a pump assembly to the container. The eyelid cleanser comprises an anionic surfactant, a non-ionic thickener and emollient, an amphoteric surfactant and at least one microbiological preservative. The surfactants include PEG-80 sorbitan laurate, sodium trideceth sulphate, PEG-150 distearate, cocamidopropylhydroxy sultaine, lauroamphocarboxy glycinate, and sodium laureth-13 carboxylate. The non-ionic thickener and emollient is PEG-15 tallow polyamine. The pump assembly is a non-propellant pump assembly for producing a foam.

A preferred method for using a dispenser for a foaming eyelid cleanser comprises depressing an actuating head on a dispenser for a foaming eyelid cleanser so that the eyelid cleanser and air in the pump assembly are mixed. the eyelid cleanser comprising an anionic surfactant, a non-ionic thickener and emollient, an amphoteric surfactant and at least one microbiological preservative, dispensing the eyelid cleanser in a container as a foam from the pump, rubbing the foamed eyelid cleanser on an eyelid and rinsing the eyelid with water.

### BRIEF DESCRIPTION OF THE DRAWINGS

FIG. 1 is a cross sectional view of the foam dispensing assembly.

FIG. 2 is a cross sectional perspective view of the foam dispensing device.

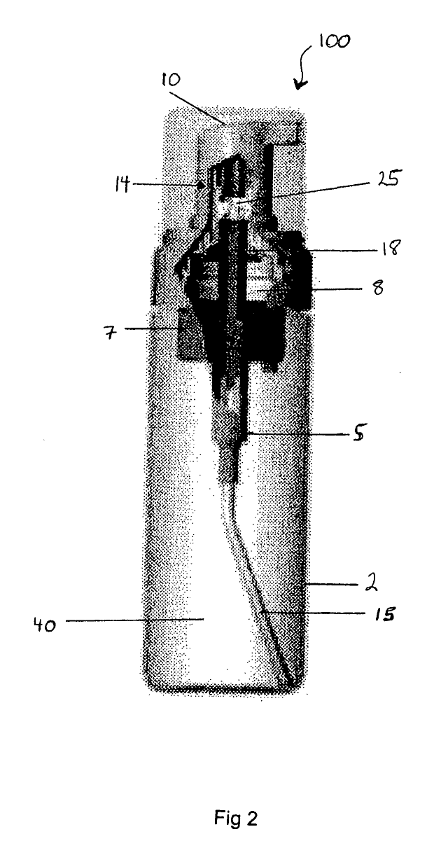

### DETAILED DESCRIPTION OF THE INVENTION

The present invention is a non-aerosol pump system for dispensing a foaming eyelid cleanser. A foam cleanser allows for the cleaning of the eyelids and ocular area while preventing the irritation caused by more flowable liquids that can accidentally enter the eye. Another advantageous feature of this invention is that the foam is dispensed in a pre-lathered format. It is well known that soaps and other surfactants must have some form of mechanical lathering before they are effective to clean. Typically, the user lathers the soap or surfactant by a rubbing action. In the present invention, the pump system dispenses the eyelid cleanser in a pre-lathered state.

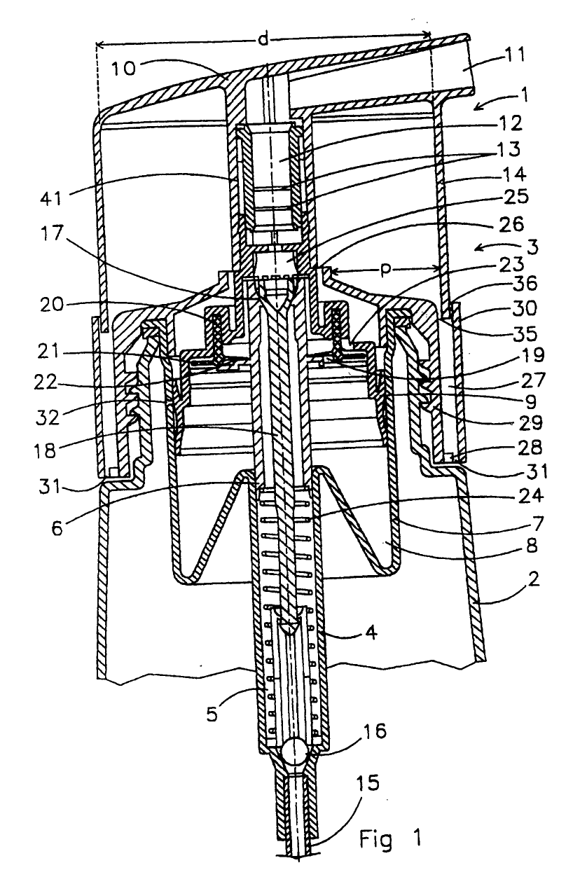

FIG. 1 shows one embodiment of the pump system of this invention illustrating the container that holds the eyelid cleanser. One preferred eyelid cleanser is an aqueous solution containing a combination of surfactants including an anionic surfactant, a non-ionic thickener, an emollient and an amphoteric surfactant. Surfactants are compounds which reduce surface tension when dissolved in water or water solutes or that reduce interfacial tension between two liquids, or between a liquid and a solid. There are three categories of surfactants—detergents, wetting agents and emulsifiers. Examples of surfactants include the polyethylene glycol or PEG compounds, anionic surfactants (a surfactant with a negative charge) and amphoteric sufactants (surfactants with the capacity to behave as either an acid or a base).

One combination of surfactants comprises PEG-80 sorbitan laurate, sodium trideceth sulfate, PEG-150 distearate, cocamidopropyl hydroxysultaine, lauroamphocarboxyglycinate and sodium laureth-13 carboxylate. Also present in the eyelid cleanser 30 composition is PEG-15 tallow polyamine in a concentration range of 0.1-0.5% by weight. This compound is a tertiary surfactant and emollient, an agent that softens and sooths.

A preservative is an agent that prolongs the useful life of a material. A biocide is a substance that kills or inhibits the growth of microorganisms such as bacteria, molds, slimes, fungi, etc. The preservative and biocide Quaternium-15 (cis 1-(3-chloroallyl)-3,5,7-triaza-1-azoniaadamantane chloride) is included in the eyelid cleanser 30. Quaternium-15 is marketed by the Dow Chemical Company of Midland, Mich. under the tradename Dowacil 200. Quaternium-15 is present in a concentration range of 0.1-0.5%. In the alternative, benzyl alcohol may be substituted in a concentration also of 0.1-0.5%. Finally, a chelating agent (a coordination compound in which a central metal ion is attached by coordinate links to two or more non-metal atoms in the same molecule) such as disodium EDTA (ethylenediaminetetraacetic acid) may be used in a concentration range of 0.01-0.10%.

In another embodiment the eyelid cleanser comprises an aqueous solution of deionized water, polysorbate 20, Quaternium-15, and potassium sorbate. This embodiment is especially effective for removing eye make-up.

A non-ionic thickening agent, a substance used to increase the viscosity of liquid mixtures and solution, and aid in maintaining the stability of the liquid by emulsifying properties may be included. The properties include reducing surface tension at the interface of the suspended particles because of the solubility properties of the molecules, referred to as a soap or detergent.

The eyelid cleanser is applied by the user to the eyelids in the form of a foam. Advantageously, the foam is produced by mechanical action rather than the use of a chemical propellant that could be an irritant to the eye. FIG. 1 shows a representative non-aerosol pump system for dispensing foaming eyelid cleanser 100 according to the invention. Such pump systems are known in the art. A pump system such as Van der Heijden, U.S. Pat. No. 6,536,629 is one preferable pump system for use with this invention.

The pump system comprises a liquid container 2 and a dispensing assembly 3. The dispensing assembly 3 is screwed onto the liquid container 2. The dispensing assembly 3 comprises a liquid pump 4 with a liquid pump chamber 5 and a liquid pump piston 6. Furthermore, there is an air pump 7 with an air pump chamber 8 and an air pump piston 9. Both pistons 6 and 9 are coupled to an actuating head 10.

The actuating head 10 comprises a dispensing opening 11 and an outlet passage 12. Two small meshes 13 are present in the outlet passage 12 in order to produce a foam. The foam is produced as the liquid is misted going through the mesh, having been pre-mixed with air. The head 10 furthermore comprises a circumferential protective cap 14.

A riser tube 15 extends close to the bottom of the liquid container 2 and serves as an inlet for the liquid pump 4. Furthermore, the inlet to the liquid pump 4 contains a non-return valve 16. The outlet passage 12 from the liquid pump 4 is situated at the location indicated by reference number 17 and can be closed off by means of a rod-like non-return valve component 18.

The outlet from the air pump 7 is situated at the location indicated by 19. A flexible sealing component is denoted by 20, comprising two annular, resilient sealing lips 21 and 22 which are used to close off and open the inlet 23 and the outlet 19 of the air pump 7. The dispensing assembly 3 is provided with restoring means which comprise a spring 24. When the foam former is in use, pressing the actuating head 10, results in the pistons 9 and 6 of the air pump 7 and the liquid pump 4, moving downwards, with the result that the volumes of the corresponding piston chambers 8 and 5, respectively, are reduced and air and liquid are dispensed to a mixing chamber 25, where they are mixed. The resulting mixture is then passed through the two small meshes 13, into the outlet passage 12, and leaves the dispensing opening 11 as a foam. The mechanical action of forcing the eyelid cleanser liquid and air through the meshes 13 causes the lathering action so that the eyelid cleanser is pre-lathered and ready to use without the rubbing action required of surfactants.

After foam has been dispensed, the actuating head 10 is released and returns to its starting position by the spring 24. During this return movement, the non-return valve 16 will open and the liquid pump chamber 5 will fill with liquid from the liquid container 2, while the air pump chamber 8 fills with air. A vent opening 32 for the liquid container 2 is used to supply air to the interior of the liquid container 2 in order to compensate for the amount of liquid which has been removed from the liquid container. Leakage water could also enter the liquid container 2 via this opening. Naturally, this is undesirable.

The pump system furthermore comprises a circumferential recess 27 with a base 28, an inner wall 29 and an outer wall 30. In the base 28 there are a plurality of outlet openings 31 which are in communication with the environment. The inner wall 29 extends as far as an annular gap 26. The gap 26 is situated between the inner wall 29 and the wall 41 of the outlet passage 12. Advantageously, the inner wall 29 according to the invention extends, as far as possible, to the vicinity of the wall 41.

During use, the protective cap 14 moves up and down in the recess 27. It is clear that the air inlet opening 23 of the air pump and the vent opening 32 in this case are situated in a space that is in communication with the environment via the gap 26 between the protective cap 14 and the inner wall 29 and the gap 35 and 36. The inner wall 29 substantially adjoins the actuating head 10 in the vicinity of the outlet passage 12. In one embodiment, the distance between the inner wall 29 and the protective cap 14 in the vicinity of that side of the inner wall 29 which is remote from the base and the internal diameter d of the protective cap 14 are also indicated in the figures.

The foregoing description is illustrative and explanatory of preferred embodiments of the invention, and variations in the size, shape, materials and other details will become apparent to those skilled in the art. It is intended that all such variations and modifications which fall within the scope or spirit for the appended claims be embraced thereby.

1. An eyelid cleanser dispensing system, the system comprising:
a foamable eyelid cleanser and a container for enclosing the eyelid cleanser; in combination with
a dispensing assembly for delivering the foamable eyelid cleanser.

2. The eyelid cleanser dispensing system of claim 1 wherein the eyelid cleanser is a foamable liquid.

3. The eyelid cleanser of claim 2 wherein the foamable liquid eyelid cleanser is an aqueous solution comprising:
(a) an anionic surfactant,
(b) a non-ionic thickener and emollient,
(c) an amphoteric surfactant; and
(d) at least one preservative.

4. The eyelid cleanser of claim 3 wherein the anionic surfactant comprises one or more of sodium trideceth sulphate and sodium laureth-13 carboxylate.

5. The eyelid cleanser of claim 3 wherein the non-ionic thickener and emollient comprises one or more of PEG-15 tallow polyamine and PEG-150 distearate.

6. The eyelid cleanser of claim 3 wherein the amphoteric surfactant comprises one or more of cocamidopropylhydroxy sultaine and lauroamphocarboxy glycinate.

7. The eyelid cleanser of claim 3 wherein the preservative is a biocide.

8. The eyelid cleanser of claim 3 wherein the preservative is Quaternium-15.

9. The eyelid cleanser of claim 2 wherein the foamable liquid eyelid cleanser comprises a surfactant mixture, the surfactant mixture comprising:
(a) PEG-80 sorbitan laurate,
(b) sodium trideceth sulphate,
(c) PEG-150-distearate,
(d) cocamidopropylhydroxy sultaine,
(e) lauroamphocarboxy glycinate, and
(f sodium laureth-13 carboxylate.

10. The eyelid cleanser of claim 8 wherein the foamable eyelid cleanser in an aqueous solution comprises:
(a) the surfactant mixture present in a concentration of 7-10%;
(b) PEG-15 tallow polyamine present in a concentration of 0.1-0.5%;
(c) at least one preservative selected from the group consisting of Quaternium-15 and benzyl alcohol, present in a concentration of 0.1-0.5% and
(e) a chelating agent present in a concentration of 0-0.1%.

11. The eyelid cleanser of claim 10 wherein the PEG-15 tallow polyamine is a surfactant emollient.

12. The eyelid cleanser of claim 10 wherein the chelating agent is disodium ethylenediaminetetraacetic acid.

13. The eyelid cleanser dispensing system of claim 1 wherein the dispensing assembly comprises a non-propellant pump for producing a foam.

14. A pump system for dispensing foaming eyelid cleanser, the system comprising:
a liquid eyelid cleanser comprising: a surfactant mixture comprising PEG-80 sorbitan laurate, sodium trideceth sulfate, PEG-150 distearate, cocamidopropyl hydroxysultaine, lauroamphocarboxyglycinate, and sodium laureth-13 carboxylate in a concentration of 7-10%; PEG-15 tallow polyamine present in a concentration of 0.1-0.5%; at least one preservative selected from the group consisting of Quaternium-15 and benzyl alcohol present in concentration of 0.1-0.5%; a chelating agent present in a concentration of 0.0-0.1% in an aqueous solution; and
a dispensing system comprising a container for enclosing the liquid eyelid cleanser in combination with a non-propellant pump foam dispensing assembly.

15. A pump system for dispensing foaming eyelid cleanser, the system comprising:
a foamable eyelid cleanser; and
a container for the eyelid cleanser in combination with a non-propellant pump dispensing assembly, the dispensing assembly comprising a liquid pump with a mixing chamber for the liquid eyelid cleanser and air, a foam producing element and an outlet passage.

16. The pump system of claim 15 comprising at least two meshes within the outlet passage.

17. The pump system claim 15 comprising:
an actuating head comprising a dispensing opening and an outlet passage containing the meshes; and
a circumferential protective cap.

18. A pump system for dispensing foaming eyelid cleanser, the system comprising:
a foamable eyelid cleanser; and
a container for the eyelid cleanser in combination with a non-propellant pump dispensing assembly, the dispensing assembly comprising a liquid pump with a mixing chamber comprising an actuating head with a dispensing opening and an outlet passage for the liquid eyelid cleanser and air, and a foam producing element comprising at least two meshes within the outlet passage.

19. A method for manufacturing a dispenser for a foaming eyelid cleanser comprising:
(a) preparing an eyelid cleanser;
(b) placing the eyelid cleanser in a container; and
(c) attaching a pump assembly to the container.

20. The method of claim 19 comprising:
(a) an anionic surfactant,
(b) a non-ionic thickener and emollient,
(c) an amphoteric surfactant,
(d) at least one microbiological preservative.

21. The method of claim 19 wherein the eyelid cleanser comprises:
a surfactant mixture comprising PEG-80 sorbitan laurate, sodium trideceth sulfate, PEG-150 distearate, cocamidopropyl hydroxysultaine, lauroamphocarboxyglycinate, and sodium laureth-13 carboxylate in a concentration of 7-10%;
PEG-15 tallow polyamine present in a concentration of 0.1-0.5%;
at least one preservative selected from the group consisting of Quaternium-15 and benzyl alcohol present in concentration of 0.1-0.5%; and
disodium ethylenediaminetetraacetic acid present in a concentration of 0.0-0.1% in an aqueous solution.

22. The method of claim 19 wherein the pump assembly is a non-propellant pump assembly for producing a foam.

23. A method for manufacturing a dispenser for a foaming eyelid cleanser comprising:
(a) preparing an eyelid cleanser comprising an anionic surfactant, a non-ionic thickener and emollient, an amphoteric surfactant, and at least one microbiological preservative;
(b) placing the eyelid cleanser in a container; and
(c) attaching a pump assembly to the container wherein the pump assembly is a non-propellant pump assembly for producing a foam.

24. A method for using a dispenser for a foaming eyelid cleanser comprising:
(a) depressing an actuating head of a dispenser for a foaming eyelid cleanser so that the eyelid cleanser and air in the pump assembly are mixed;
(b) dispensing the eyelid cleanser as a foam from the pump;
(c) rubbing the foamed eyelid cleanser on an eyelid; and
(d) rinsing the eyelid with water.

25. The method of claim 24 wherein the foaming eyelid cleanser comprises
(a) an anionic surfactant,
(b) a non-ionic thickener and emollient,
(c) an amphoteric surfactant; and
(d) at least one microbiological preservative.

26. The method of claim 24 wherein the pump assembly is a non-propellant pump assembly for producing a foam.
